SEQUENCE LISTING (1 sequence)

SEQ ID NO: 1 - DNA, 19 bp, Artificial Sequence
  Synthetic
cctatagtgagtcgtatta

(19) United States
(12) Patent Application Publication
(10) Pub. No.: US 2006/0149484 A1
(43) Pub. Date: Jul. 6, 2006

(54) Absolute quantitation of nucleic acids by rt-pcr

(76) Inventor: Normand E. Allaire, Sharon, MA (US)

Correspondence Address: STERNE, KESSLER, GOLDSTEIN & FOX, P.L.L.C., 1100 NEW YORK AVE., N.W., WASHINGTON, DC, 20005, US

(21) Appl. No.: 10/534,689

(22) PCT Filed: Nov. 14, 2003
(86) PCT No.: PCT/US03/36522

§ 371 Date: Jan. 23, 2006

Related U.S. Application Data
(63) Continuation of application No. 10/294,781, filed on Nov. 14, 2002.

(51) Int. Cl. C12Q 1/68; G06F 19/00

(52) U.S. Cl. 702/20; 435/6

(57) ABSTRACT

A method for obtaining a cRNA for use in generating calibration data, e.g., a standard curve, for absolute quantitation of RNA by RT-PCR is disclosed. The method includes the steps of: providing a synthetic oligonucleotide comprising an amplicon, a promoter sequence located 3′ relative to the amplicon; synthesizing complementary RNA (cRNA) by in vitro transcription of the synthetic oligonucleotide; quantitatively assaying the cRNA by an independent method; and generating calibration data using a known quantity of the cRNA.

### TECHNICAL FIELD

The invention relates to molecular biology. More particularly, it relates to real-time PCR methods and absolute quantitation of gene expression.

### BACKGROUND

Basic PCR technology applies to amplification of a DNA sequence of interest. In reverse transcriptase PCR (RT-PCR) a reverse transcription step is added to the PCR protocol. This adapts basic PCR methodology for detection and quantitation of specific mRNA transcripts. Thus, RT-PCR is suitable for measuring and comparing gene expression levels. Examples of useful comparisons include expression in different tissue types in an individual organism, in the same tissue type among different organisms, and in the same tissue type in response to experimental treatment(s). Quantitation can be relative, i.e., expressed in terms of fold-difference between samples, or absolute, i.e., in terms of actual amount of RNA.

Historically, following synthesis of cDNA in a reverse transcription step, PCR was run to an appropriate end point in an amplification step, and then quantitation of reaction product was carried out in a separate detection, i.e., assay, step. In a variation of RT-PCR known as “real time” PCR (or “kinetic PCR”), the amplification step and detection step are combined. The cycle-by-cycle increase in the amount of PCR product is quantified in real time. This is accomplished by including a “probe” along with conventional forward and reverse primers in the amplification reaction. The probe, which hybridizes within the amplified sequence, typically is about 20-25 nucleotides in length. The commercially available TaqMan® probes (Applied Biosystems, Foster City) Calif.) include a fluorescent reporter moiety (dye) at the 5′ terminus and a quencher moiety (dye) at the 3′ terminus. In the intact probe, fluorescence of the reporter is strongly suppressed through internal quenching by a quencher moiety. As the exonuclease action of the advancing Taq polymerase digests the hybridized probe, the reporter is unquenched, resulting in fluorescence, which is detected and quantified.

Quantitation of mRNA by real-time PCR can be relative or absolute. In either case, quantitation of mRNA in a sample is by reference to an appropriate standard curve. In the case of standard curves for relative quantitation, quantity is expressed relative to a basis sample, which is often called the “calibrator.” For the experimental samples, target quantity is determined from the standard curve and divided by the value of the calibrator. Thus, the calibrator becomes the 1× sample, and all other quantities are expressed as an n-fold difference relative to the calibrator. For example, in a study of drug effects on gene expression, the untreated control would serve as a suitable calibrator. Because the experimental quantity is divided by the calibrator quantity, the standard curve unit, e.g., fluorescence intensity, drops out. This means that for relative quantitation, any source of RNA or DNA containing the target sequence can be used to create a standard curve, following preparation of a suitable dilution series.

In contrast, absolute quantitation of mRNA by real-time PCR requires a standard in which the absolute quantity of an RNA containing the target sequence is known independently. Typically, a plasmid containing a cDNA containing the target sequence must be obtained. A restriction fragment of the plasmid that contains the cDNA (and no other open reading frames) is gel-purified and reverse-transcribed. The A₂₆₀ of the resulting cRNA measured, and the cRNA is used to prepare a standard curve. Complementary RNA copy number is calculated from the A260 and the molecular weight of the mRNA. This presents little difficulty where expression of one gene, or a small number of genes, is assayed repetitively, for example, in clinical testing of viral load in HIV patients. However, in situations where large numbers of different targets must be quantitated by real-time PCR, this approach to absolute quantitation is so time-consuming and laborious that it becomes impractical.

### SUMMARY OF THE INVENTION

The invention provides a method for obtaining a cRNA for use in generating calibration data, e.g., a standard curve, for absolute quantitation of RNA by RT-PCR. The method includes the steps of: (a) providing a synthetic oligonucleotide comprising an amplicon, a promoter sequence located 3′ relative to the amplicon; (b) synthesizing complementary RNA (cRNA) by in vitro transcription of the synthetic oligonueleotide; (c) quantitatively assaying the cRNA by an independent method; and (d) generating calibration data using a known quantity of the cRNA.

Preferably the promoter sequence is a bacteriophage promoter sequence. An example of a promoter sequence useful in the invention is a T7 promoter sequence, e.g.: 5′CCTATAGTGAGTCGTATrA 3′ (SEQ ID NO:1). The synthetic oligonucleotide optionally includes a 5′ flanking sequence of 2-20, preferably 8-12, nucleotides adjacent to the amplicon. In some embodiments of the invention, the 5′ flanking sequence contains 5-20 consecutive thymine residues, i.e., an oligo d(T) region. The synthetic oligonucleotide optionally includes a 3′ flanking sequence of 2 to 20, preferably 8-12, nucleotides between the amplicon and the promoter region.

The length of the amplicon preferably is 30 to 70 nucleotides, and more preferably, 40 to 60 nucleotides. The length of the synthetic oligonucleotide preferably is 60 to 140 nucleotides, more preferably 70 to 130 nucleotides, 80 to 120 nucleotides, or 90 to 110 nucleotides.

The invention also features an RT-PCR method for determining the abundance of specific nucleic acid molecules, e.g., a specific RNA transcript, in a test sample. The method includes the steps of: (a) providing a synthetic oligonucleotide comprising an amplicon and a promoter sequence located 3′ relative to the amplicon; (b) synthesizing cRNA by in vitro transcription of the oligonucleotide; (c) producing a dilution series using the cRNA; (d) synthesizing single-stranded cDNA by reverse transcription of the cRNA; (e) generating PCR calibration data using the cDNA; (g) obtaining PCR test sample data from the test sample; and (h) comparing the PCR test sample data to the PCR calibration data. In preferred embodiments of the invention, the cRNA is assayed quantitatively by an independent method, e.g., A₂₆₀, and mixed with heterologous RNA, e.g., yeast total RNA, prior to synthesis of the single-stranded cDNA.

As used herein, “amplicon” means a specific preselected nucleotide sequence amplified in a PCR reaction.

As used herein, “flanking sequence” means a nucleotide sequence adjacent to an amplicon in a synthetic oligonucleotide. A 5′ flanking sequence is located 5′ relative to the amplicon. A 3′ flanking sequence is located 3′ relative to the amplicon.

As used herein, “PCR calibration data” means PCR data generated using known quantities of a nucleotide sequence corresponding to a target sequence, against which test data will be compared for purposes of quantitation. Calibration data may be relative or absolute.

As used herein, “standard curve” means a plot (usually semi-logarithmic) of calibration data.

As used herein, “synthetic oligonucleotide” means a nucleic acid containing a specific sequence of nucleotides produced by synthetic organic chemistry rather than by enzymatic polymerization in a living cell.

As used herein, “target sequence” means a sequence to be assayed, e.g., a sequence in a gene, cDNA or RNA of interest.

Unless otherwise defined, all technical and scientific terms used herein have the same meaning as commonly understood by one of ordinary skill in the art to which the invention belongs. In case of conflict, the present application, including definitions, will control. All publications, patents and other references mentioned herein are incorporated by reference.

The materials, methods and examples presented below are illustrative only, and not intended to be limiting. Other features and advantages of the invention will be apparent from the detailed description and from the claims.

### BRIEF DESCRIPTION OF THE DRAWINGS

FIG. 1 is a schematic illustration of the general structure of a synthetic oligonucleotide for use in the invention. The structure illustrated in FIG. 1 includes 5′ and 3′ flanking sequences, which are optional.

FIG. 2 is a standard curve for absolute quantitation of mRNA by RT-PCR. Purified cRNA was subjected to six successive 1:10 serial dilutions and then placed (“spiked”) into a yeast total RNA background. Each dilution of cRNA was used for synthesis of cDNA, which was used as a starting template for RT-PCR. The RT-PCR was performed in quadruplicate (R²=0.9980). The Ct value (Cycle threshold) is the output for the RT-PCR assay. The Ct is the cycle number recorded when the reaction becomes exponential. When this happens then the equation is true Ct=2^{initial amount}. A Ct was generated for unknowns and standards. Ct values for unknowns were then compared to the Ct standard curve.

FIG. 3 is a histogram showing the results of absolute quantitation of mRNA according to the invention. Messenger RNA copy number was determined in tissue from rat lung, liver, kidney, heart, spleen, thymus, embryo and brain, based on the standard curve shown in FIG. 2.

### DETAILED DESCRIPTION OF THE INVENTION

In the present invention, a novel combination of known elements or steps has been assembled into a simplified method for obtaining a cRNA for use in generating a PCR calibration data for absolute quantitation of RNA by RT-PCR. Enhanced efficiency offered by the method makes it practical to perform absolute quantitation of mRNA by real-time RT-PCR in a high throughput situation involving numerous different target sequences. Thus, the method is useful in situations such as basic biological research and drug discovery programs.

The invention advantageously avoids any need to obtain a plasmid containing a cDNA containing the target sequence. In addition, the invention avoids the need to generate and purify a restriction fragment of the plasmid that contains the cDNA (and no other open reading frames). This simplification results from utilization of a synthetic oligonucleotide in combination with an in vitro transcription step. Following the in vitro transcription step, conventional real-time PCR methodology can be applied with or without modification. For a detailed discussion of various aspects of steps following the in vitro transcription step, see generally, PCR Protocols in Molecular Toxicology, 1997, CRC Press. See also, Sambrook et al., Molecular Cloning, A Laboratory Manual, 1989, Cold Spring Harbor Press.

If the RNA obtained by in vitro transcription is going to be used in generating PCR calibration data for absolute quantitation of RNA, a sample of RNA obtained in the in vitro transcription step is assayed quatitatively. This can be done, for example, by measuring the absorbance of a solution of the RNA at 260 nm (A₂₆₀). The A₂₆₀ value can be converted to an RNA concentration value, which can be converted to a copy number, based on the calculated molecular weight of the cRNA molecule involved.

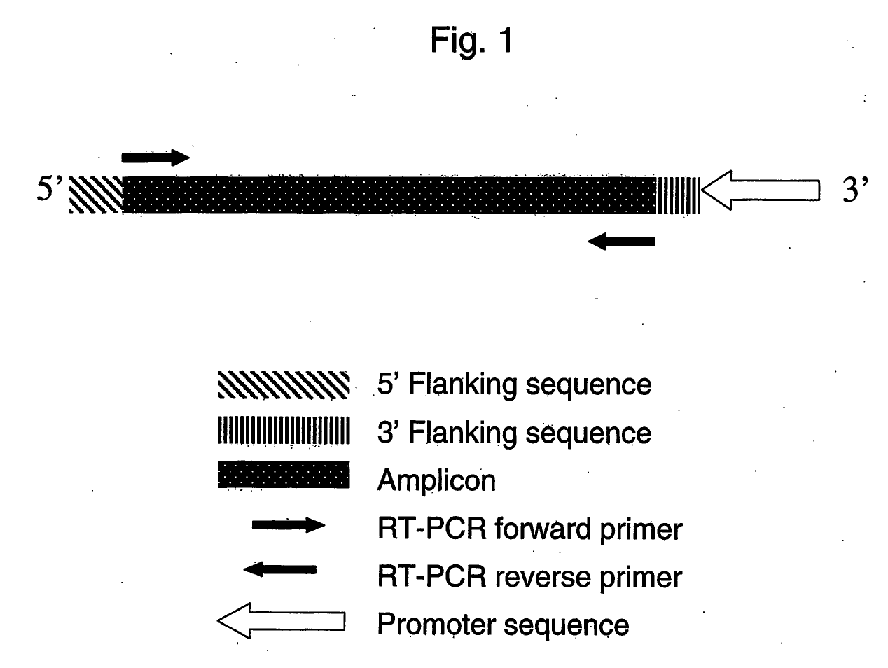

Design of the synthetic oligonucleotide is within ordinary skill in the art. In general, the synthetic oligonucleotide contains an amplicon, promoter sequence and optional amplicon-flanking sequences (FIG. 1). The nucleotide sequence of the synthetic oligonucleotide will depend on considerations including the amplicon sequence, sequences flanking the amplicon in the target sequence, and the choice of promoter sequence.

The length of the amplicon is not critical. Preferably the amplicon length is in the range of 30 to 70 nucleotides. More preferably, it is from 40 to 60 nucleotides. Amplification of a particular amplicon in a PCR system is achieved by the design and synthesis of an appropriate forward primer and an appropriate reverse primer. The design and synthesis of PCR primers is well known in the art, with primer designing software, reagents and instrumentation being commercially available. An example of such commercial software is Primer Express® (Applied Biosystems, Foster City, Calif.).

A 5′ flanking sequence, a 3′ flanking sequence, or both, can be included adjacent to the amplicon. Preferably, both are included. The flanking sequences, if present, may differ from each other in sequence and length. The length of the optional flanking sequence(s) is not critical. Preferably, each flanking sequence is from 2 to 20 nucleotides, and more preferably from 8 to 12 nucleotides. The nucleotide sequence of the flanking sequences is not critical. For example, it can be designed to hybridize to the target gene, but such complementarity is not necessary. In some embodiments of the invention, the 5′ flanking sequence in the synthetic oligonucleotide includes, or consists of, a poly T tail. This results in a corresponding poly A tail in the subsequently-produced cRNA, which is useful for priming the reverse transcription reaction. The length of a suitable poly T (poly A) tail is from 5 to 20 nucleotides, with about 16 nucleotides being preferred.

A promoter sequence is incorporated in the synthetic oligonucleotide. Any promoter sequence that functions effectively under the reaction conditions employed in the in vitro transcription reaction can be used. Bacteriophage promoter sequences often are used for in vitro transcription reactions. Specific examples of promoters useful in the invention are T7, SF6 and T3 promoters. A preferred T7 promoter sequence is a T7 promoter sequence, e.g.: 5′CCTATAGTFGAGTCGTATTA 3′ (SEQ ID NO:1). Those of skill in the art will recognize that the termini of promoters are not always crisply defined, and that minor changes in naturally occurring promoter sequences often can be made while retaining (or even improving) promoter function. A suitable promoter sequence can be selected and incorporated by one of skill in the art without undue experimentation. Promoter sequences suitable for use in the invention are commercially available.

The overall length of the synthetic oligonucleotide, i.e., including amplicon, promoter, and optional amplicon-flanking sequence(s) must be short enough to permit chemical synthesis and long enough to permit in vitro transcription. In most cases, the length will be in the range of 60 to 140 nucleotides. Preferably, the length will be in the range of 70 to 130, 80 to 120, or 90 to 110 nucleotides.

The exact sequence of the synthetic oligonucleotide is designed according to particular choices with respect to the subsequence components discussed above. Once designed, the synthetic oligonucleotide can be synthesized by one of ordinary skill in the art using known methods, materials and instrumentation. Synthetic oligonucleotides suitable for use in the present invention can be obtained from commercial sources, e.g., Biosearch Technologies, Inc. (Novato, Calif.) and Invitrogen, Inc. (Carlsbad, Calif.).

It is to be understood that the present invention involves generally applicable analytical methodology. Thus, the invention is not specific to any particular target sequence, amplicon, or synthetic oligonucleotide.

Synthetic oligonucleotides for use in the present invention can be obtained by any suitable synthetic method. Methods, materials and instrumentation for synthesis of oligonucleotides having a predetermined sequence of well over 100 nucleotides are known in the art. See, e.g., Cheng et al., 2002, Nucleic Acids Research 30 (18) e93. Custom-synthesized oligonucleotides for use in the invention can be obtained from commercial sources, e.g., Biosearch Technologies Inc. (Novato, Calif.); Invitrogen (Carlsbad, Calif.). Purification of the synthetic oligonucleotides can be achieved using conventional technology, e.g., reverse phase HPLC. Sequences of the synthetic oligonucleotides can be confirmed by standard sequence methods.

The in vitro transcription step in the present invention can be carried out using known methods and materials. Achievement of desirable yield may involve optimized reaction conditions for RNA synthesis in the presence of high nucleotide and polymerase concentration. Reagents and kits for carrying out the in vitro transcription step are commercially available. A suitable commercial kit is the MEGAshortscript™ T7 Kit (Ambion, Inc., Austin, Tex.; cat. #1354). A partial single-stranded template can be used for the in vitro transcription reaction. For example, a primer complementary to the T7 promoter region can be used to create a short double-stranded region to which the T7 polymerase binds and initiates transcription. Alternatively, a double-stranded template can be used. For example, one could anneal a primer to the promoter region of the synthetic oligonucleotide and extend it with a DNA polymerase, e.g., a Klenow fragment. Then the resulting double-stranded template could be purified and used for in vitro transcription. In a variation of the double-stranded template approach, a complete second strand complementary to the synthetic oligonucleotide could be synthesized and annealed. The cRNA product should be obtained as a single species. This can be verified, e.g., by gel electrophoresis.

Accurate serial dilution of cRNA is desirable for the production of reliable standard curves. Regarding preparation and characterization of RNA standards, see generally, Collins et al., 1995, Analytical Biochemistry 226:120-129. Preferably, a carrier RNA is used in serial dilutions. A preferred carrier RNA is yeast total RNA at a concentration of about 25 ng/ml (Ambion, Inc., Austin, Tex.; cat. #7118).

In methods according to the invention, synthesis of cDNA can be achieved in a conventional reverse transcription reaction. Methods and materials for reverse transcriptase reactions are known in the art. See, e.g., Sambrook et al. (supra). Kits for reverse transcription reactions are available from various commercial vendors, e.g., High Capacity cDNA Archive Elite (Applied Biosystems, Inc., Foster City, Calif.).

The invention is further illustrated by the following examples. The examples are provided solely for purposes of illustration. They are not to be construed as limiting the scope or content of the invention in any way.

### EXAMPLES

Primer, Probe Design and Oligonucleotide Templates

Taqman forward and reverse primers and 5′ FAM labeled MGB probes were designed from Affymetrix consensus sequences using Primer Express® (Applied Biosystems). Oligonucleotide templates for in vitro transcriptions reactions were designed by adding 10 base pairs of gene specific sequence to the 5′ and 3′ ends of the amplicon, followed by the addition of a T7 promotor region consisting of 5′CCTATAGTGAGTCGTAJTA 3′ (SEQ ID NO:1) external to the 3′ 10 base pairs.

In Vitro Transcriptions Using Synthetic Oligonucleotides

In vitro transcription reactions using partially single stranded oligonucleotide templates were performed using a commercial kit (T7-MEGAshortscript Kit™, Ambion Inc., Austin, Tex.). Partially single stranded templates were prepared by annealing the a T7 primer (5′AATTTAATACGACTCACACTATAGG 3′) which is complementary only to the T7 promotor region of the synthetic oligonucleotide template in 10 mM Tris-HCl (pH 8.0), 1 mM EDTA, 0.1 M NaCl) in equimolar amounts (20 μM), heated to 95° C. for five minutes and cooled to room temperature. The partially single stranded template (1.5 μM reaction concentration) was used in the in vitro transcription reaction at 37° C. for 4 hours, according to manufacturer's protocol (Ambion Inc., Austin, Tex.). Oligonucleotide template DNA was removed by the addition of 2U of RNase-free DNase 1 (Ambion Inc., Austin, Tex.) for 15 minutes at 37° C. Reactions were terminated by the addition of 20 μL formamide (50% v/v), vortexing, and heating at 95° C. for 3 minutes. In vitro transcription reactions were purified using a commercial kit in accordance with the vendor's recommended prototcol (MEGAclear Kit™, Ambion).

Concentrations of cRNA were determined by measurement of uv absorbance at 260 nm. Quality of the cRNA was evaluated by running a 150 ng aliquot on 10% TBE urea polyacrylamide (BioRad, Inc., Hercules, Calif.).

Synthesis of cDNA for Standard Curves

Complementary RNA preparations that produced single bands of correct size on sizing gels were added to (spiked into) yeast sheared RNA. First, the cRNAs were subjected to eight successive serial dilutions of 1:10. An aliquot of each dilution was added to a background of yeast sheared RNA (1 μg/μL) (Ambion Inc., Austin, Tex.). Complementary DNAs were produced in eight corresponding reverse transcription reactions (100 μL) performed using 10 μg spiked yeast total (sheared) RNA as template. The reverse transcription reactions were carried out using a commercial kit in accordance with the vendor's recommended protocols (High-Capacity cDNA Archive Kit™, Applied Biosystems).

Synthesis of cDNA from Experimental Samples

Rat total RNA from lung, liver, kidney, heart, spleen, thymus, embryo and brain (10 μg) (Ambion, Inc.) were used as templates for cDNA synthesis reactions using a reverse transcription kit in accordance with protocols supplied by the kit vendor (High Capacity cDNA Archive Kit™, Applied Biosystems). A 1 μl aliquot from each cDNA synthesis reaction (100 ng total RNA starting template) was used as an experimental sample for each quantitative RT-PCR reaction.

Taqman Thermal Cycling

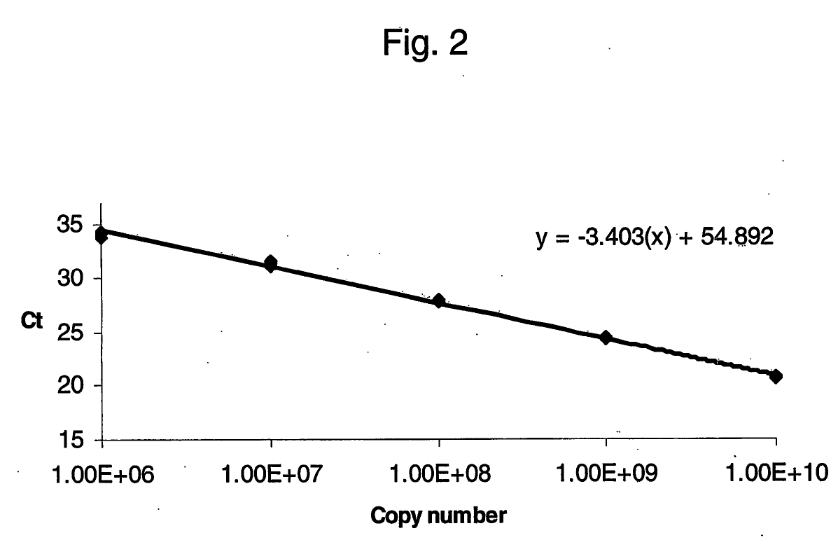

Quadruplicate PCR reactions for standards and for experimental samples were mixed in a 96-well plate, and then transferred to a 384-well optical plate (Applied Biosystems, Foster City, Calif.). Real-time reactions were cycled in a model 7900HT thermal cycler (Applied Biosystems, Foster City, Calif.). Thermal cycler conditions were as follows: 50° C. for 2 minutes (uracil N-deglycosylase digest); 95° C. for 10 minutes (activation of Taq thermostable polymerase); and 40 cycles of 95° C. for 15 seconds and 60° C. for 60 seconds with 900 nM forward and reverse primers, 200 nM Taqman MGB probe, and 1× Universal master mix (Applied Biosystems). In each reaction well, fluorescence emission was measured every seven seconds for the length of the run. A standard curve was generated from PCR data obtained using the standards (FIG. 2).

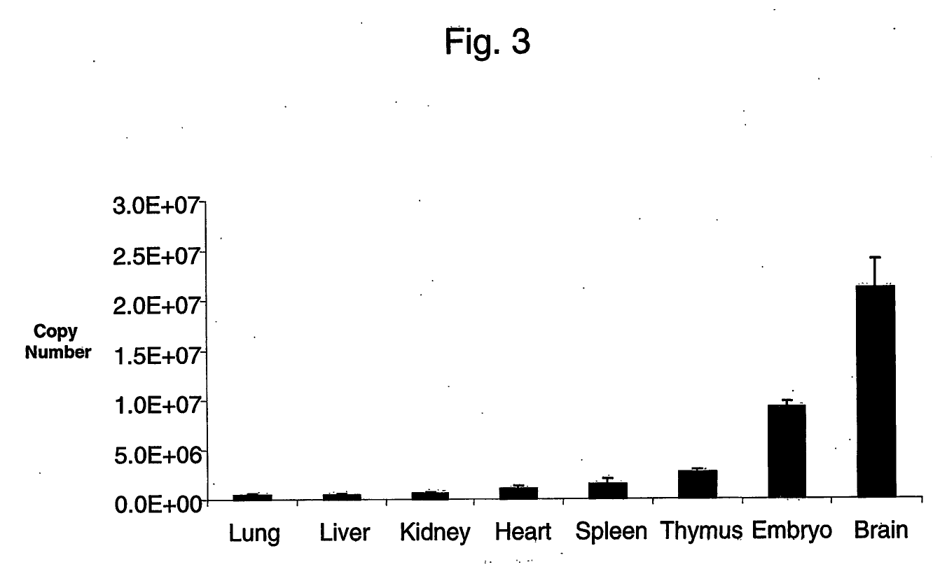

Transcript quantities were determined for each experimental sample by comparison to the absolute cRNA standard curves, using Sequence Detection Software (Applied Biosystems). Copy numbers for the various experimental samples were obtained by comparison of experimental sample PCR data to the standard curve. Copy number results are summarized in FIG. 3.

Other embodiments are within the following claims.

1-18. (canceled)

19. A calibrator for absolute quantitation of target RNA by RT-PCR comprising a single cRNA species synthesized by reverse transcription from a synthetic oligonucleotide and quantitatively assayed by an independent method, wherein said synthetic oligonucleotide comprises an amplicon and a promoter sequence located 3′ relative to the amplicon.

20. The calibrator of claim 19, wherein said promoter sequence is a bacteriophage promoter sequence.

21. The calibrator of claim 20, wherein said promoter sequence is a T7 promoter sequence.

22. The calibrator of claim 21, wherein said T7 promoter sequence is CCTATAGTGAGTCGTATTA (SEQ ID NO:1).

23. The calibrator of claim 19, wherein said synthetic oligonucleotide further comprises a flanking sequence of 2-20 nucleotides adjacent to the amplicon.

24. The calibrator of claim 23, wherein said flanking sequence is 5′ and adjacent to the amplicon.

25. The calibrator of claim 23, wherein said flanking sequence is 3′ and adjacent to the amplicon.

26. The calibrator of claim 23, wherein synthetic oligonucleotide further comprises both a 5′ and a 3′ flanking sequence adjacent to the amplicon.

27. The calibrator of claim 23, wherein said flanking sequence is 8 to 12 nucleotides in length.

28. The calibrator of claim 19, wherein said amplicon is 30 to 70 nucleotides in length.

29. The calibrator of claim 28, wherein said amplicon is 40 to 60 nucleotides in length.

30. The calibrator of claim 19, wherein said synthetic oligonucleotide is 60 to 140 nucleotides in length.

31. The calibrator of claim 30, wherein said synthetic oligonucleotide is 70 to 130 nucleotides in length.

32. The calibrator of claim 31, wherein said synthetic oligonucleotide is 80 to 120 nucleotides in length.

33. The calibrator of claim 32, wherein said synthetic oligonucleotide is 90 to 110 nucleotides in length.

34. The calibrator of claim 19, further comprising heterologous RNA.

35. The calibrator of claim 34, wherein said heterologous RNA is total RNA.

36. The calibrator of claim 35, wherein said heterologous RNA is yeast total RNA.

37. The calibrator of claim 19, wherein said cRNA species is MGB.

38. The calibrator of claim 19, wherein said cRNA is assayed quantitatively by measuring its absorbance at 260 nm.

39. A kit comprising the calibrator of claim 19.

40. The kit of claim 39 comprising more than one aliquot of said cRNA species.

41. The kit of claim 40, wherein each said aliquot comprises successive serial 1:10 dilutions of said cRNA species.
